(19) United States
(12) Patent Application Publication
(10) Pub. No.: US 2021/0035692 A1
(43) Pub. Date: Feb. 4, 2021

(54) Tool for Development of a Validated Frailty Risk Score

(71) Applicant: Patient Pattern, Inc., Buffalo, NY (US)

(72) Inventor: Steven Buslovich, Buffalo, NY (US)

(21) Appl. No.: 16/527,764

(22) Filed: Jul. 31, 2019

(51) Int. Cl. G16H 50/30; G16H 10/60; G16H 10/20; G06F 16/22

(52) U.S. Cl. CPC G16H 50/30; G06F 16/22; G16H 10/20; G16H 10/60

(57) ABSTRACT

A system for displaying a frailty index is disclosed. A platform in data communication with a test computer can determine a first frailty index score from health survey data. A database accessible by the platform stores the data and the frailty index score. The platform can generate a report based on the health survey data, and display the report on a display computer.

### TECHNICAL FIELD

This disclosure relates to electronic medical systems for determining the frailty of a patient through both new and existing data.

### BACKGROUND

Frailty is an under-disclosed chronic condition and geriatric syndrome that causes age-related vulnerability to poor outcomes. Creating a mathematical model of patients' frailty is difficult to assess in practice. Therefore, to manage populations experiencing high Frailty Index Scores across the care continuum, a software approach to calculating frailty is preferred.

Frailty can be measured in relation to the accumulation of deficits using a Frailty Index. A Frailty Index can be developed from most ageing databases. The principle is to count deficits in health (which can be symptoms, signs, diseases, disabilities or laboratory, radiographic or electrocardiographic abnormalities) on the grounds that the more deficits a person has, the more likely that person is to be frail. The index is often expressed as a ratio of deficits present to the total number of deficits considered. For example, if 40 deficits were considered, and 10 were present in a given person, that person's frailty index would be 10/40=0.25. When calculating a Frailty Index Score the deficits are also referred to herein as components of the Frailty Index Score.

Presently, frailty is calculated by hand through a comprehensive geriatric assessment which can take upwards of two hours to complete. Expert geriatric practitioners are less common than other types of medical practitioners and are often limited by time constraints, making it difficult to get expert assessment on the results of these tests. The data from MDS and OASIS surveys are not easily translatable into a frailty score. It is presently not practical to spend resources converting survey results into frailty scores for each patient.

Further, the assessments are not scalable or available electronically, causing there to be less comprehensive and less comparable data on the frailty of patients. The assessments are presently not able to be retrospectively analyzed to determine the accuracy of frailty assessments. As there is no gold standard for frailty assessment, predictive validation is an important method of validating any approach to frailty operationalization.

Frail individuals typically rely on a complex system of providers and supports in housing, food/nutrition, transportation, socialization, etc. A simple medical practice-based initiative to maximize quality of life and function for these individuals falls short. Unfortunately, although we spend significant dollars on medical concerns, the United States healthcare system delivers the least impressive outcomes of the major advanced countries when comparing costs, access, and longevity. One of the most significant barriers to effectiveness is a lack of acknowledgement that frail individuals are experiencing a chronic progressive condition that has its own pathway, similar to other diseases. Failure to recognize this leads to misdiagnoses, over treatment, and promises of outcomes that cannot be delivered. Costs skyrocket due to ineffective surgeries, prolonged hospital stays, even institutionalization among 50 percent of old frail individuals. Increased medical expenditures have diminished benefit and in some cases actually trend toward harm.

### SUMMARY

Frailty is a chronic condition of physiological losses across multiple body systems that ultimately affects a patient's function and cognition. Patients with a poor Frailty Index Score are more vulnerable to increased dependency, poor outcomes, and/or mortality, due to their degree of frailty. These patient may require more or less attention depending on the value of putting resources toward a patient with such a Frailty Index Score or Frailty Index Score chart.

Understanding frailty—and how it's measured—allows for a proactive approach to early interventions. Using frailty to predict risk will improve patient outcomes, as well as help patients and families make treatment decisions. Frailty accurately predicts the risk for re-hospitalization, potential for recovery and/or return to baseline.

A Frailty Index Score can be utilized in a variety of ways. If a patient transitions from one care setting to another care setting, for example, from an acute-care hospital to a skilled nursing facility. The care setting transition necessitates a change in the clinical care delivery team as the patient moves from one healthcare facility to another. At the point of transition, the patient's Frailty Index Score can be determined effectively via software through a digital screening module and associated analytics. The resultant Frailty Index Score can be used by the incoming clinical care delivery team to accurately and quickly create an appropriate care plan for the patient, resulting in better and more consistent healthcare outcomes.

Further, the new patient admission impacts the clinical and operational resources required by the new facility to provide ongoing care for its patient population—for example, available nursing staff, available medical supplies, and available accommodations. At the point of transition, the patient's Frailty Index Score can be determined rapidly via software through a digital screening module and associated analytics. The resultant Frailty Index Score can be used by the new facility to assess whether additional clinical and operational resources must be obtained to meet patient care needs and determine the facility's ongoing ability to admit new patients with various levels of acute illness.

If a patient in a healthcare facility experiences a change in medical condition. The facility staff wishes to communicate with the patient's family with respect to this change in condition in a way that will help the family better understand its loved-one's current medical state and future prognosis. The patient's Frailty Index Score can be determined rapidly via Patient Pattern's software through a digital screening module and associated analytics. The Frailty Index Score can then be used, via software's graphical interfaces, as a basis of candid and understandable discussion among healthcare providers and family members as to the patient's current and anticipated future healthcare needs and condition. Frailty Index Scores can be used to help optimize clinical outcomes and provide patient-centered care by aligning patient and family expectations with associated risks for poor outcomes and decline.

The Frailty Index Score can be used by a healthcare facility's quality assurance team to internally audit and compare/contrast patient healthcare outcomes achieved by the healthcare facility for a specified time-period to identify overall trends in the facility's success metrics and to evaluate the performance of various providers and methodologies of treatment for patients with similar and contrasting Frailty Index Scores.

The Pharmaceutical industry can utilize Frailty Index Scores in clinical trials for purposes of drug design, efficacy testing and pre-market deployment evaluation. Post-market analysis of outcomes can incorporate Frailty Index Scores as a measure of clinical risk to compare outcomes and drug efficacy/performance. Using Frailty Index Scores to demonstrate improvements in clinical outcomes.

Electronic Health Record and data exchange protocols can deploy Frailty Index Scores to provide an assessment of a patient and a difference in scores can be used to represent a potential change in clinical condition of a patient. This can trigger evaluation and other clinical interventions.

Since insurance and other payment companies may find Frailty Index Scores beneficial to overall treatment, the use of a Frailty Index can be reimbursed through various payment models. The payment models include Accountable Care Organizations, Program for All-Inclusive Care of the Elderly, Medicare Advantage products, institutional Special Needs Plans, Dual Special Needs Plans, Chronic Special Needs Plans, Direct Contracting plans, and other similar reimbursement models where providers of care take on financial risk. The Frailty Index Score can be used by the family or provider to determine cost effectiveness.

Frailty is not measured by age or diagnosis. There are three components that contribute to the measurement of frailty: psychological changes, physical declines, and social disruptions. Psychological changes include cognition, mood, and motivation of the patient. Physical declines include nutrition, mobility, strength, energy, and physical activity of the patient. Social disruptions including social contact and support of the patient and the amount of isolation. The frailty of a patient can also be used to determine how many resources should be spent on a patient by a care facility. By tracking the patient's frailty over time, the facility can get better insight into the needs and best practices for treating the patient.

Accordingly, it is an object of the invention to create a longitudinal collection of frailty data accessible through an electronic health system.

To further the object of this invention, a Frailty Index may be designed such that the Frailty Index can be determined through analysis of pre-existing clinical data sets. These data sets might include MDS or OASIS data sets.

Alternatively, the survey may be a web-based smart assessment. The web-based smart assessment is designed such that non-geriatric trained health care professionals can complete the assessment at the patient's bedside in real-time. Further, the web-based smart assessment may be specifically tailored to a Frailty Index. The web-based smart assessment may be carried out on a mobile computing device, such as a tablet, and scored to create a Frailty Index in real-time.

In one object of the invention the Frailty Index is created through a collection of scores of different frailty components. The frailty components could be, for example, happiness, eyesight, drowsiness, touch, light sensitivity, incidents, appetite, hearing, stress, family, heartbeat, memory, fashion, and strength. One skilled in the art would appreciate that other components could be added or subtracted from this list to create the collection.

Each component is given a score, based on how important to the frailty of the patient the component is. For example, memory may be 2, while strength is 1. In one object of the invention the patient is give a score for each of the components, and the scores are added up to determine the overall frailty of the patient.

Since many patients may already had surveys conducted with data indicative of the patient's frailty, it is an object of this invention to create a system that automatically retrieves this data and converts it into a Frailty Index Score. This may be done by first standardizing all the patient data. For example, an MDS survey may ask about a patient's happiness, and score that value between 0 and 27. The computer would read that score and assign it a score based on the Frailty Index, for example either 0 if less than 10, 1 if greater than 9 and less than 15, and 2 is greater than 14. This would be done for each component of the Frailty index that is also a part of the MDS survey. The components that are in the Frailty Index that are not in the MDS survey, could be deleted, and the Frailty Index Score would be related to only the included components. Alternatively, a medical practitioner could take a second survey of the patient to fill in the blanks. The questions could come from a computer, which automatically prompts which questions need to be answered.

After the data has been standardized, it is sent to a platform, which may be a Frailty Index Platform. The platform receives the data, applies the rules engine and determines the Frailty Index Score based on the standardized data. The Frailty Index Score can then be stored on the platform, or on a database off the platform. A display computer, which may be that of a physician or medical facility, has access to the database or platform to retrieve the Frailty Index Score data.

It is an object of the invention to plot the Frailty Index Score data longitudinally for easier viewing at the medical facility. As medical professionals take smart surveys of the patients, this data could be updated in real-time. This allows the medical facility to see changes in a patient's frailty to better treat and care for the patient.

When the patient has taken a frailty test, or the medical facility has accessed the frailty assessment an automatic payment may be sent to the company providing the frailty service. Alternatively, an automatic payment, or payment set-up may be done based on time with the program, for example monthly billing. In another object of the invention, upon the generation of a report, the platform may automatically send a bill. This bill could also be sent to an insurance provider, such as Medicare.

In an object of the invention a medical facility can graph the data longitudinally to see how each patient compares to the norm for their age, or otherwise sorted comparison. Alternatively, the platform can plot the data for the medical facility, and the medical facility will receive a plot from the platform. The platform can also show which patients are improving, which are deteriorating, retrospectively assess the frailty of patients, integrate the Frailty Index Score into other software systems and/or provide patient snapshots to show the specific components that go into each patients Frailty Index Score.

### BRIEF DESCRIPTION OF THE DRAWINGS

FIG. 1 is an embodiment of the system for determining a Frailty Index Score.

FIG. 2 is an embodiment of the system for determining a Frailty Index Score.

FIG. 3 is a depiction of raw data.

FIG. 4A is a screenshot of an embodiment of a Frailty Index Score Report.

FIG. 4B is a screenshot of an embodiment of a Frailty Index Score Report.

FIG. 5A is a sample Frailty Index.

FIG. 5B is a sample calculation of a Frailty Index Score.

FIG. 6A is an embodiment of converting data to Frailty Index data.

FIG. 6B is an embodiment of converting data to Frailty Index data.

### DETAILED DESCRIPTION

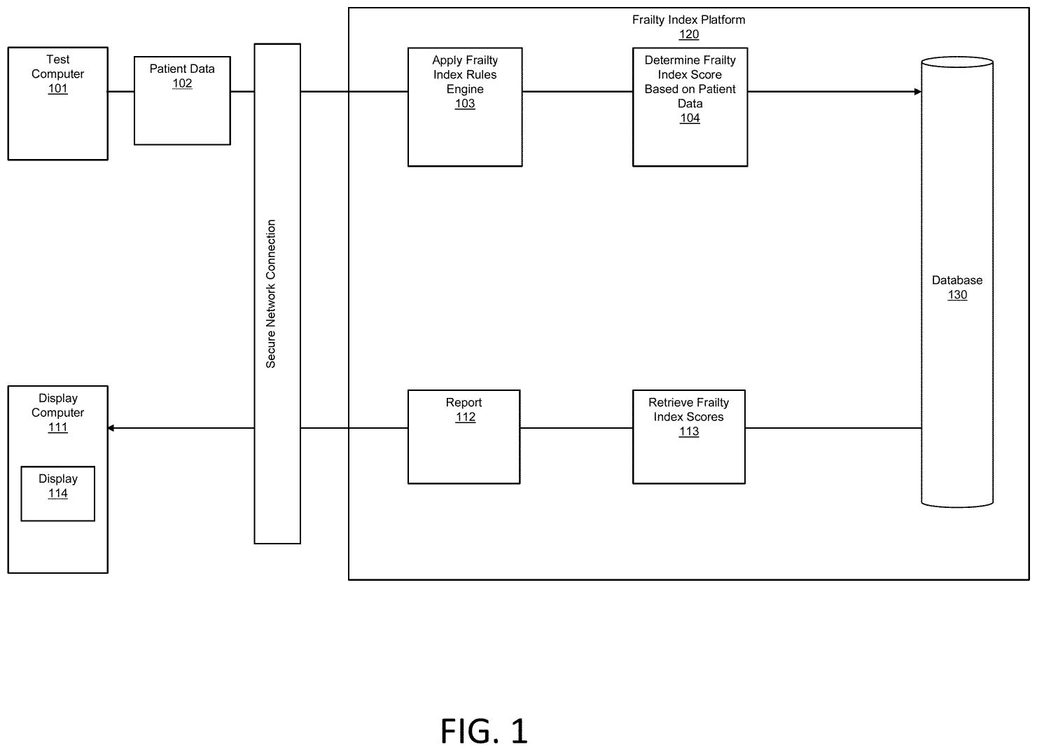

FIG. 1 depicts a system for determining a Frailty Index Score of a patient. A Test computer 101 transmits patient data 102 to a Frailty Index Platform 120. The test computer 101 could be a computer used by a medical practitioner at a patient's bedside. The test computer 101 could also be a computer storing patient data from a medical survey such as MDS or OASIS.

If the medical professional is taking the survey on the test computer 101, the patient data 102 is indicative of the answers to the survey questions given by the medical professional. The patient data 102 is sent to a Frailty Index Platform 120. The patient data 102 may be sent via a secure network connection. This may be done via the internet.

The Frailty Index Platform 120 has software that will receive the patient data 102 from test computer 101. The Frailty Index Platform also has software which applies the Frailty Index rules engine 103 to the patient data 102. The Frailty Index rules engine 103 determines a Frailty Index Score. The Frailty Index Score 104 is indicative of a patients Frailty. The Frailty Index Score 104 is stored on a database 130. The database 130 may be a part of the same electronic device as the Frailty Index Platform 120, or could be separate and connected via a network connection, for example, the internet.

The Frailty Index Platform 120 can also create a report 112 from the Frailty Index Score 104. The report 112 may be a graphical representation of the patient data 102 or the report 112 could be a graphical representation of a plethora of patients' Frailty Index Scores 104. One skilled in the art would appreciate that the report 112 could take numerous different forms depending on the viewer's desire.

A display computer 111 has a network connection to the Frailty Index Platform 120. The display computer 111 may be accessed by a medical facility who reviews the frailty of a collection of patients, or the display computer 111 could be accessed by an individual, such as the family of the patient. The display computer 111 has a display 114 where the Frailty Index Score is shown. Additionally, the display 114 may show patient data 102 or the report 112. In one object of the invention the display computer 111 would make a query to the Frailty Index Platform 120, which informs the Frailty Index Platform to retrieve the Frailty Index Scores from the database 130.

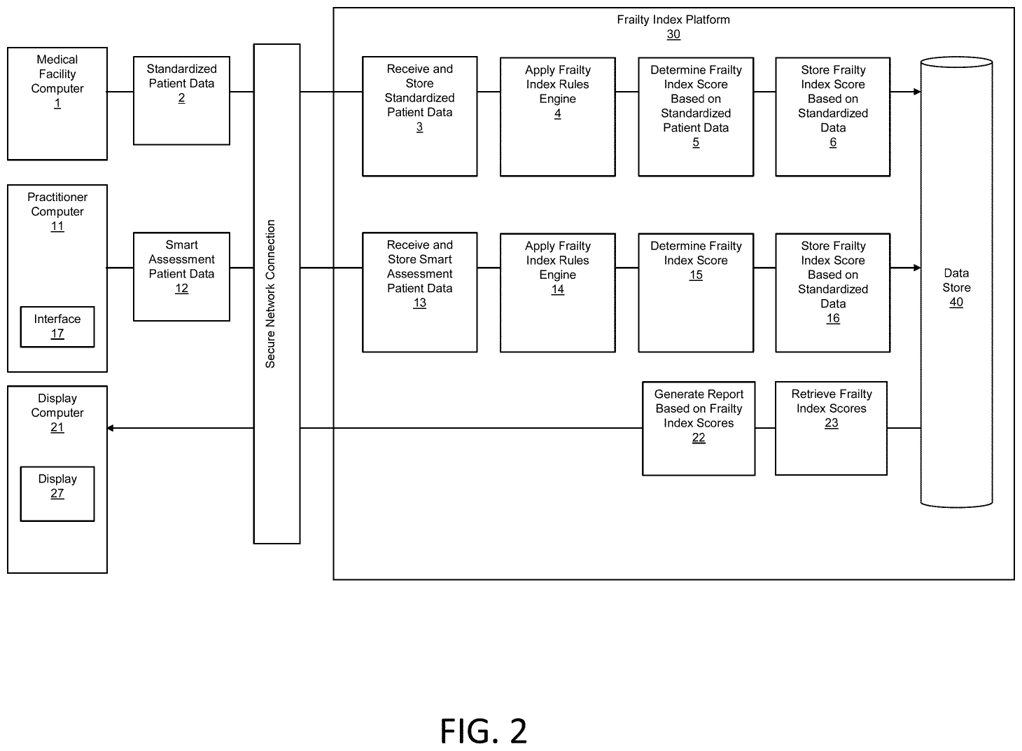

FIG. 2 depicts a system configured to generate a Frailty Index Score report. The medical facility computer 1 contains standardized patient data 2. The standardized patient data 2 might be from a medical survey standardized by the government. For example, MDS or OASIS. The standardized surveys are not always sufficient to determine a Frailty Index Score. The surveys also have different scoring systems from the Frailty Index. Therefore, it is an object of the invention to automate the conversion of the standardized patient data 2 into a Frailty Index Score.

The Frailty Index Platform 30 first receives and stores the standardized patient data as shown in step 3. The standardized patient data 2 can be stored on the Frailty Index Platform 30 or a database separate from the platform. Next, the Frailty Index Platform 30 applies the Frailty Index rules engine to the standardized patient data 2. The software applying the Frailty Index Rules Engine 4 is configured to translate the results of medical surveys and convert them into data which can accurately create a Frailty Index Score. For example, an MDS survey may have a scale of 1-29, while the Frailty Index Score may have a scale of 0-2. Accordingly, the software applying the Frailty Index Rules Engine 4 will convert the MDS survey scale into an appropriate Frailty Index scale.

After the Frailty Index Rules have been applied software 5 running on the Frailty Index Platform 30 will determine a Frailty Index Score based on the standardized patient data. The score may be a ratio of the total possible points versus the points the patient had. The score may also be depicted in other ways. In another object of the invention, the score may be broken into different categories.

Once the score is calculated, software 6 running on the Frailty Index Platform stores the score. The score may be stored on a database separate from the Frailty Index Platform 30, or on a data store 40 within the Frailty Index Platform 30.

Still referring to FIG. 2, a second method of a calculated Frailty Index Score is provided, via practitioner computer 11. In one object of the invention, a practitioner, such as a nurse or doctor, can conduct the survey on the bedside of the patient via the practitioner computer 11. The practitioner computer 11 has an interface 17 which would display the question to be asked and allow the practitioner to select the answer. The smart survey is designed to fill in as many components of the Frailty Index Score as possible with each questions. For example, if the first questions is how much help does the patient need getting out of a chair, and the answer is fully dependent, the survey does not need to ask about climbing stairs. The smart assessment will eventually create smart assessment patient data 12.

The practitioner computer 11 is in data communication with the Frailty Index Platform 30. The Frailty Index Platform 30 has software 13 to receive and store the smart assessment patient data 12. The Frailty Index Platform 30 may receive the smart assessment patient data 12 when the survey is complete, or it may receive the data in real time as each questions is inputted by the practitioner.

The Frailty Index Platform 30 has software 14 for applying the Frailty Index Rules engine 14 to the smart assessment patient data. This, for example, will turn an answered question, such as fully dependent on getting out of a chair, into a component score. In another object of the invention, the smart assessment may determine the frailty score of each question on the practitioner computer 11, and thus the smart assessment patient data 12 will already be in proper Frailty Index format when received by the Frailty Index Platform 30.

Similar to with the standardized patient data 2, the Frailty Index Platform 30 has software 15 for determining the Frailty Index Score from the smart assessment patient data 12. The score may be a ratio of the total possible points versus the points the patient had. The score may also be depicted in other ways. In another object of the invention, the score may be broken into different categories.

Once the score is calculated, software 16 running on the Frailty Index Platform 30 stores the score. The score may be stored on a database separate from the Frailty Index Platform 30, or on a data store 40 within the Frailty Index Platform 30.

A display computer 21 is in data communication with the Frailty Index Platform 30, for receiving a Frailty Index Score report. The report may include a graphical showing of the patient's Frailty Index Score over time. The datastore 40 or other data storage system as one skilled in the art would appreciate may store each patients Frailty Index Score data from past examinations. Seeing the Frailty Index Score over time of a patient may be valuable to assess if the treatment plan of the patient is working successfully, or to analyze trends and assume what other patients will need in the future.

In another object of the invention the report could contain data on a plurality of patients. This would allow a medical facility to see the frailty of a collection of its patients to determine appropriate staffing, or to create models, such as how many people are expected to pass in the upcoming months.

The display computer 21 may send a query to the Frailty Index Platform 30 asking for specific data. The Frailty Index Platform 30 has software 23 for retrieving said data from the datastore 40. The data may be Frailty Index Scores or raw data recorded from a survey or smart assessment. The Frailty Index Platform has software 22 for generating a report on the data. This may include graphing the data, sorting the data, or other data manipulation.

The display computer 21 may be connected to the Frailty Index Platform 30 through a secure network connection. The generated report can be sent via the secure network connection to the display computer 21. The display computer has a display 27 which shows the report to a user or users. This could be a medical practitioner, family of the patient, or other user.

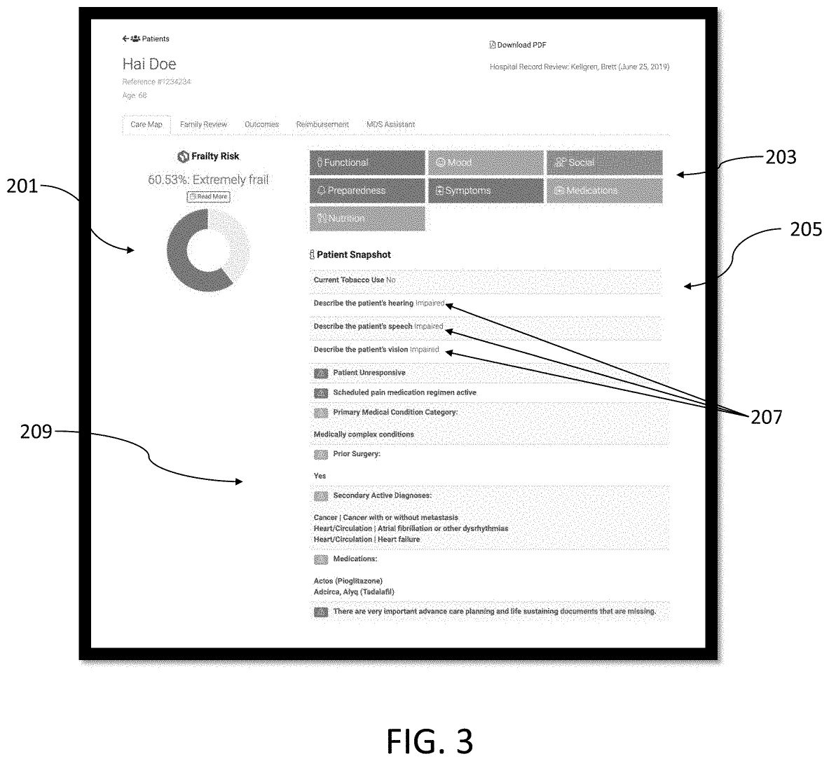

FIG. 3 is a screenshot depicting one object of the invention. A Frailty Index Score 201 of a single patient is displayed in graphical form. Tabs 203 shows the patient's frailty in different sorted categories to get a closer understanding of this particular patient. A patient snapshot 205 shows raw data of the patient, and also shows if certain patient data is missing or incomplete. Survey answers 207 are shown which give insight to specifically why the score was achieved.

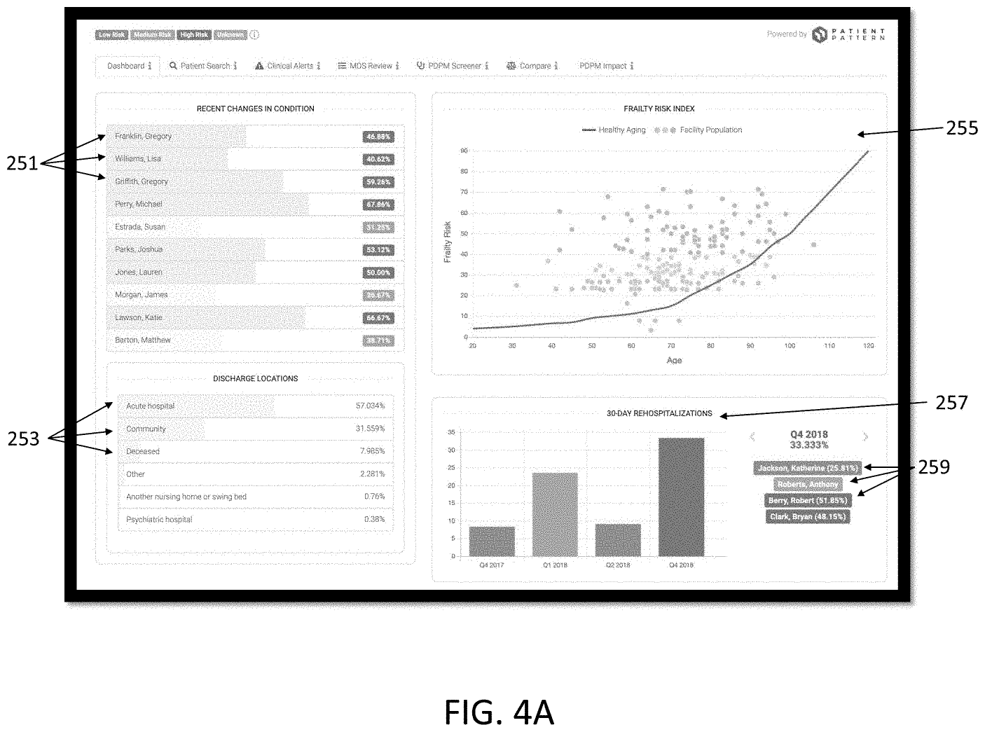

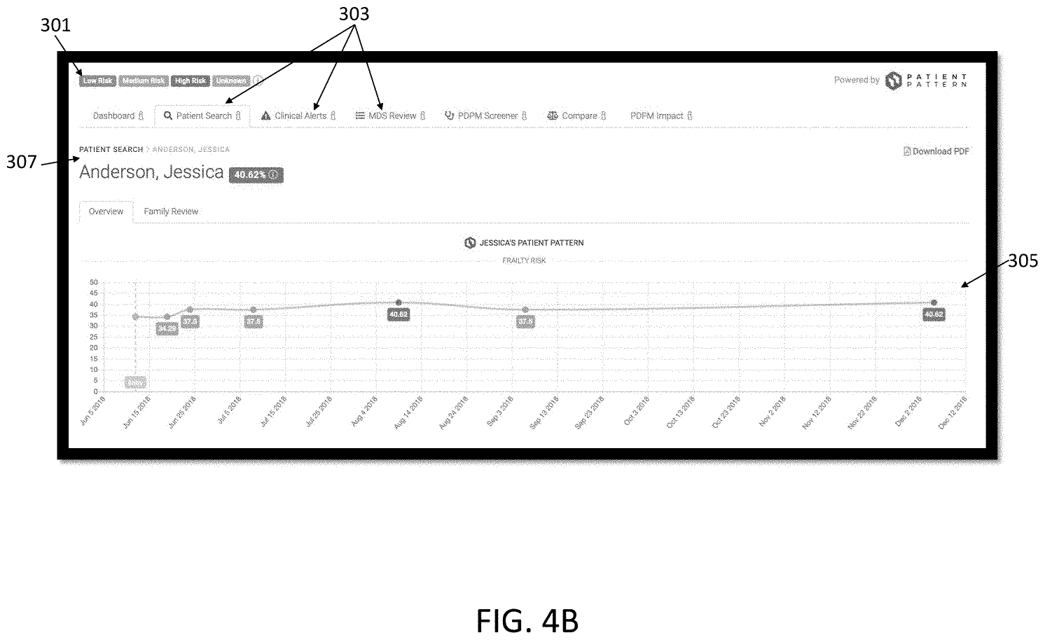

FIG. 4A is a screenshot of one object of the invention showing a plurality of patients Frailty Risk Indices. Recent changes in condition 251 are shown. A medical facility may note the recent changes to pay more close attention to what may be the cause, or to take better care of deteriorating patients. The graph 255 shows patients frailty risk versus their age. Of course, older patients will become naturally frailer, which is depicted in the healthy aging curve. The curve and plot can be used to determine how at risk general population of the facility is. It is also useful to plot each datapoint on a longitudinal graph to evaluate how a medical facility is performing.

FIG. 4A also shows discharge locations of patients 253. This may be interesting to families looking to put their loved one in a medical facility. This data may come from the standardized patient data, smart assessments, or other data sent to the Frailty Index Platform. Graph 257 shows 30-day re-hospitalizations. Other graphs may be selected or shown depending on the desired data by the user. Names 259 are shown to specify which patients have been more at risk for certain events.

FIG. 4B shows the patient pattern of a single patient 307. Markers 301 show what risk the patient currently has. Tabs 303 allow the user to view different data indicative of this patient health. For example, one tab is the MDS review which may show the patients raw MDS survey results. Graph 305 shows different Frailty Index Scores of the patient over time, graphed longitudinally. This shows the deterioration, or lack thereof, of the patient's frailty. By graphing this data longitudinally, medical facilities will receive more data on the correlation between a patient's frailty and their overall health. The medical facility can also track the patient's frailty over time for example, to see how certain treatments affect the patient or to see if a decline is inevitable. The longitudinal graph also allows for surveys conducted in the past to be entered and properly plotted. This allows for retrospective data analysis on frailty and patients.

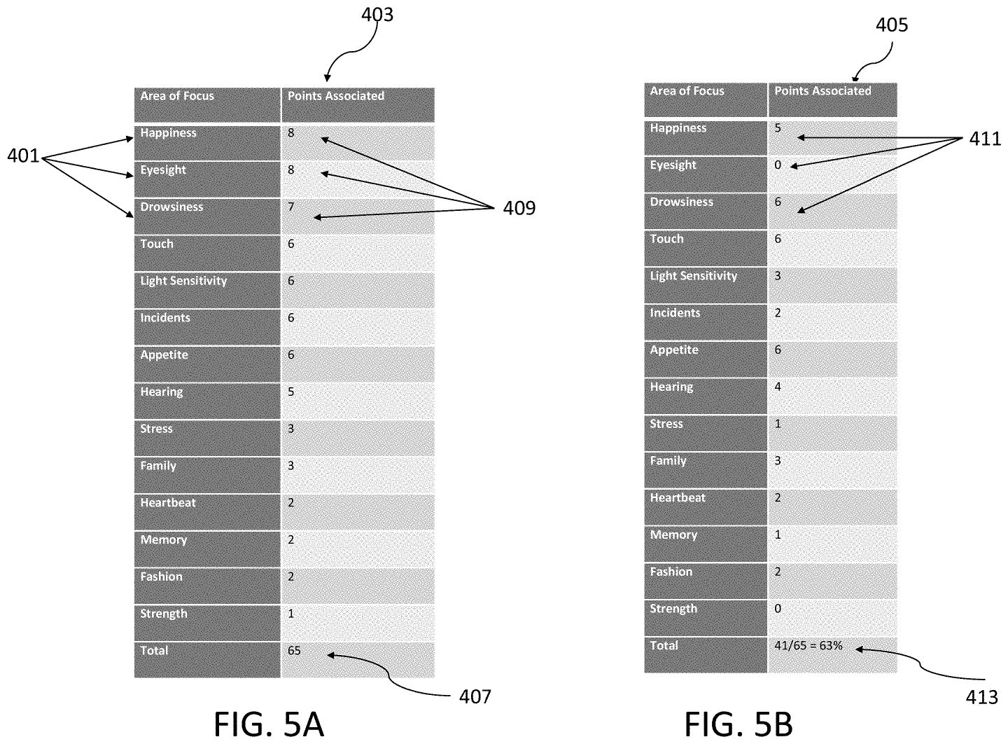

FIG. 5A shows a sample Frailty Index 403. Components 401 are listed which each are an indication of the patient's frailty. Component scores 409 show how much weight is given to each component. The total number 407 is the base of the Frailty Index Score. One skilled in the art would understand that a Frailty Index Score may comprise many different components, some of which are not mentioned in this application. FIG. 5B shows a sample Frailty Index Score. Each component value 409 has been given an actual component score 411. The total of those scores is then added, and the base total 407 is the denominator. The Frailty Index Score 413 is shown. One skilled in the art would appreciate that the calculation of a Frailty Index may depend on different components 401 and different component values 409.

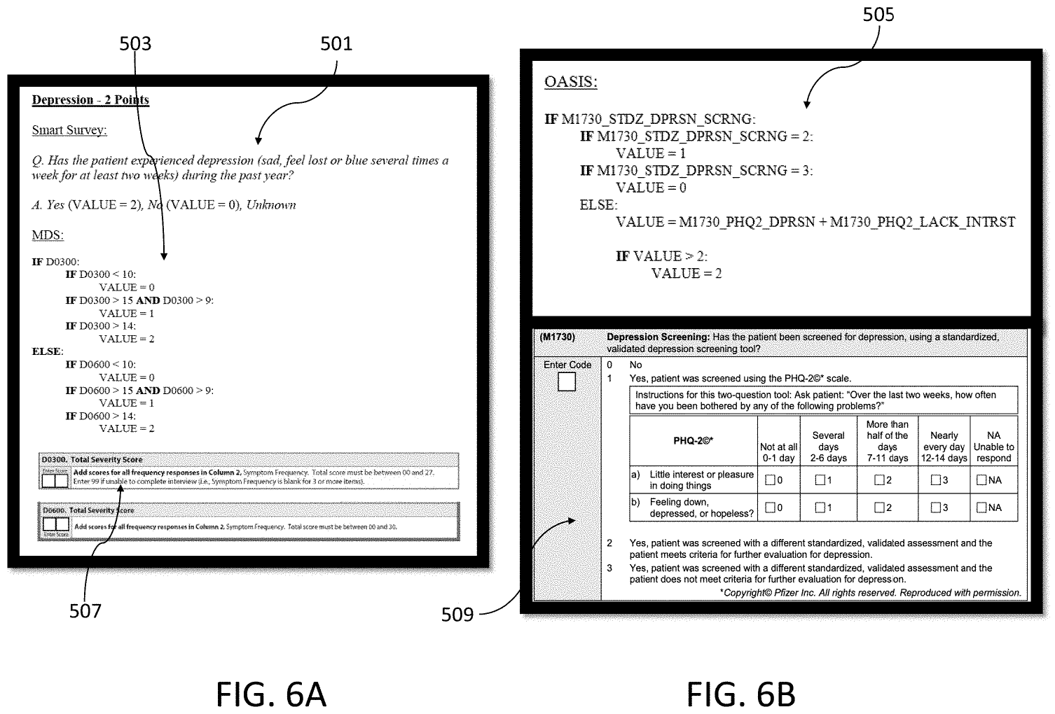

FIG. 6A shows one object of the invention to compare a smart survey and the conversion of MDS to Frailty Index. Survey question 501 is assigned different Component scores based on the possible answers to the question. Code 503 shows an example of how MDS survey results are converted into Frailty Index Scores. The survey question 507 asks for a certain range, and that range is then converted into the Frailty Index range. Similarly, FIG. 6B shows how an OASIS survey question is converted into Frailty Index Scores. Question 509 asks for a certain range, and the OASIS data is converted into Frailty Index data.

Although the invention has been described with reference to a particular arrangement of parts, features and the like, these are not intended to exhaust all possible arrangements or features, and indeed many other modifications and variations will be ascertainable to those of skill in the art.

What is claimed is:

1. A system for displaying a frailty index comprising:
a test computer,
a platform in data communication with the test computer,
software executing on said platform for determining a first frailty index score from health survey data,
a database accessible by the platform for storing the health survey data,
software executing on said platform for generating a report based on the health survey data, and
a display, in data communication with the platform, which displays the report.

2. The system of claim 1 wherein the report displays the frailty index score on a longitudinal graph.

3. The system of claim 2 wherein the report is indicative of the frailty index score of a plurality of patients.

4. The system of claim 2 wherein the report is indicative of only one patient and said patient's Frailty Index Score over time.

5. The system of claim 1 wherein the test computer is a medical facility computer wherein the health survey data is a standardized health survey.

6. The system of claim 5 further comprising:
a practitioner computer having a smart assessment, wherein said smart assessment generates smart assessment data,
software executing on the platform for determining a second frailty index score from the smart assessment data,
software executing on said platform for generating a report based on the smart assessment data
wherein the database stores smart assessment data.

7. The system of claim 6 wherein the report is based on the smart assessment data.

8. The system of claim 7 wherein the report displays the first frailty index score and the second frailty index score on a longitudinal graph.

9. A system for displaying a frailty index comprising:
a test computer,
a platform in data communication with the test computer, wherein the test computer has an interface which performs a smart assessment wherein the smart assessment generates smart assessment data,
software executing on said platform for determining a first frailty index score from the smart assessment data,
a database accessible by the platform for storing the smart assessment data,
software executing on said platform for generating a report based on the smart assessment data, and
a display, in data communication with the platform, which displays the report.

10. The system of claim 9 wherein the test computer is a laptop or tablet.

11. They system of claim 9 wherein the smart assessment is performed on a patient and the smart assessment data is received by the platform in real-time.

12. The system of claim 9 wherein the smart assessment is composed of a series of questions wherein an answer to a first question determines a subsequent question.

13. The system of claim 12 wherein the subsequent question is a consecutive question.

14. The system of claim 9 wherein the smart assessment is composed of a series of questions wherein an answer to a first question determines the omission of a second question.

15. The system of claim 9 wherein the display is an electronic medical record.

16. The system of claim 1 wherein the test computer is in a community, home care, practice, or hospice setting.
